(19) United States
(12) Patent Application Publication
(10) Pub. No.: US 2005/0208008 A1
(43) Pub. Date: Sep. 22, 2005

(54) Detergent composition comprising at least one polymer comprising at least one heteroatom and at least one oil

(76) Inventors: Eric Parris, Clichy (FR); Serge Restle, Saint Prix (FR)

Correspondence Address: Thomas L. Irving;FINNEGAN, HENDERSON, FARABOW, GARRETT & DUNNER, L.L.P., 1300 I Street, N.W., Washington, DC, 20005-3315, US

(21) Appl. No.: 11/014,835

(22) Filed: Dec. 20, 2004

Related U.S. Application Data
(60) Provisional application No. 60/532,920, filed on Dec. 30, 2003.

(30) Foreign Application Priority Data
Dec. 19, 2003 (FR) 03 51143

(51) Int. Cl. A61K 7/06; A61K 7/11

(52) U.S. Cl. 424/70.11

(57) ABSTRACT

The present disclosure relates to a detergent composition comprising, in a cosmetically acceptable medium, at least one polymer comprising a heteroatom and a fatty chain in combination with at least one oil and at least one surfactant chosen from anionic and nonionic detergent surfactants. This composition may be used as shampoo and makes it possible to obtain good hair conditioning properties. The present disclosure also relates to a cosmetic treatment process, for example, a hair treatment process, employing the composition disclosed herein.

This application claims benefit of U.S. Provisional Application No. 60/532,920, filed Dec. 30, 2003.

The present disclosure relates to a composition comprising at least one polymer comprising at least one heteroatom in combination with at least one oil. The present disclosure also relates to a detergent composition or a cosmetic treatment process, for example, a hair treatment process, employing the composition disclosed herein.

In the field of cosmetics, at least one aim is, for example, to improve the conditioning of the hair. As used herein, the term “conditioning” is understood to mean properties of easy disentangling, of sheen, of softness to the touch, of sleekness, and of smoothness (visual and to the touch).

The use of a polymer with an end fatty chain and with a repeat unit comprising at least one heteroatom is known in cosmetics and, for example, in the field of makeup, as is disclosed in French Patent Application No. 2 817 743. In cosmetic compositions, the fatty phase of which is gelled by such polymers, has also been disclosed in French Patent Application No. 2 796 270, which discloses, for example, solid lipstick compositions in the form of a stick.

U.S. Pat. No. 5,783,657 illustrates certain types of polymers which may participate in the composition of the formulations of the present disclosure. Nevertheless, there is an absence of detergent cosmetic compositions.

Accordingly, the present disclosure relates to using at least one polymer having a fatty chain chosen from end and pendant fatty chains and with a repeat unit comprising at least one heteroatom in combination with at least one oil in detergent composition for hair treatment. With the present disclosure, it may be possible to obtain a very good hair conditioning effect.

Without being committed to any one theory, it would appear that, under these conditions, the deposit of oil on the hair may be significantly increased, resulting in an increased effectiveness. However, this improvement may be made without having a heavy greasy feel, which is usually the case when the amount of oil is increased.

Furthermore, this conditioning effect may be persistent with regard to rinsing.

An embodiment of the present disclosure is a liquid detergent composition comprising, in a cosmetically acceptable aqueous medium, at least one surfactant chosen from anionic and nonionic detergent surfactants, at least one oil and at least one polymer with a weight-average molecular mass of less than or equal to 1,000,000 comprising a) a polymer backbone having hydrocarbonaceous repeat units provided with at least one heteroatom and b) at least one optionally functionalized fatty chain chosen from pendant and end fatty chains having from 6 to 120 carbon atoms bonded to the hydrocarbonaceous units, and from 70% to 95% by weight of water, relative to the total weight of the composition.

Another embodiment of the present disclosure is a liquid detergent composition comprising, in a cosmetically acceptable medium, at least one oil, at least one surfactant chosen from anionic and nonionic detergent surfactants, at least one polymer with a weight-average molecular mass of less than or equal to 1,000,000 comprising a) a polymer backbone having hydrocarbonaceous repeat units provided with at least one heteroatom and b) at least one optionally functionalized fatty chain chosen from pendent and end fatty chains having from 6 to 120 carbon atoms bonded to these hydrocarbonaceous units, and at least one amphoteric surfactant.

A further embodiment of the present disclosure is a process for the treatment of keratinous substances employing a composition according to the disclosure.

Other subject-matters, characteristics, aspects and advantages of the disclosure will become even more clearly apparent on reading the description and the various examples which follow.

According to the present disclosure, the detergent composition comprises at least one polymer defined below in combination with at least one oil and at least one surfactant chosen from anionic and nonionic detergent surfactants.

According to another embodiment of the disclosure, the at least one oil is, for example, prethickened by the above polymer e.g., a structuring polymer, that is to say that the at least one oil and the at least one polymer may be mixed before introduction into the composition.

The at least one oil/at least one polymer of the disclosure comprises a heteroatom ratio by weight, for example, greater than or equal to 50/50, such as greater than or equal to 60/40, for example, ranging from 60/40 to 99/1 and further for example, from 80/20 to 99/1.

The at least one oil, for example prethickened, is dispersed in the form of particles in the aqueous composition. The oil particles, for example, exhibit a number-average primary size ranging from 1 μm to 100 μm, such as, from 5 μm to 30 μm and further for example, from 10 μm to 20 μm.

Within the meaning of the present disclosure, the term “particle primary size” is understood to mean the maximum dimension which it is possible to measure between two diametrically opposite points of an individual particle. The size may be determined, for example, by transmission electron microscopy or from the measurement of the specific surface by the BET method or else via a laser particle sizer.

The structuring polymer of the composition of the disclosure may be a solid which is nondeformable at ambient temperature (25° C.) and atmospheric pressure (760 mm of Hg). The structuring polymer may be insoluble in water or the aqueous phase; it may be capable of structuring (i.e., thickening) the at least one oil. For example, the structuring polymer does not crystallize and the structuring of the liquid fatty phase may be due to hydrogen bond interactions between two polymer molecules and/or between the molecules of the at least one polymer and the molecules of the liquid fatty phase. For example, the structuring polymer may not have an ionic group.

As used herein, the term “functionalized chains” is understood to mean, an alkyl chain comprising at least one functional or reactive groups chosen, for example, from amide, hydroxyl, ether, oxyalkylene and polyoxyalkylene, halogen, ester, siloxane and polysiloxane groups, the halogen groups including fluorinated and perfluorinated groups. In addition, the hydrogen atoms of at least one fatty chain may be at least partially substituted by fluorine atoms.

According to the disclosure, the at least one functionalized fatty chain may be bonded directly to the polymer backbone or via an ester functional group or a perfluorinated group.

As used herein, the term “polymer” is understood to mean a compound having at least 2 repeat units and, for example, at least 3 repeat units which are identical.

As used herein, the term “hydrocarbonaceous repeat unit” is understood to mean a unit comprising from 2 to 80 carbon atoms and, for example, from 2 to 60 carbon atoms, carrying hydrogen atoms and optionally oxygen atoms, which can be linear, branched or cyclic and saturated or unsaturated. These units each additionally comprise from one to several advantageously non-pendant heteroatoms which are found in the polymer backbone. These heteroatoms are chosen from nitrogen, sulfur and phosphorus atoms and their combinations, optionally in combination with at least one oxygen atom. For example, the units comprise at least one nitrogen atom, such as, a non-pendant nitrogen atom. In addition, these units may comprise a carbonyl group.

The units with a heteroatom are, for example, amide units, forming a backbone of the polyamide type, or carbamate and/or urea units, forming a polyurethane, polyurea and/or polyurea-urethane backbone. These units may be amide units. The pendant chains may be bonded directly to at least one of the heteroatoms of the polymer backbone. According to one embodiment, the first polymer comprises a polyamide backbone. In addition, the end chains are bonded to the polymer backbone via a bonding group chosen from ether, amine, urea, urethane, thioether, thioester, thiourea and thiourethane groups and a single bond.

The polymer may comprise units chosen from silicone units and oxyalkylenated units between the hydrocarbonaceous units.

In addition, the at least one polymer of the composition of the disclosure comprises a total number of fatty chains ranging from 40% to 98% of the total number of the units with the at least one heteroatom and the at least one fatty chain and for example, from 50% to 95%. The nature and the proportion of the units with the at least one heteroatom depends on the nature of the liquid fatty phase and is, for example, similar to the nature (polar or nonpolar) of the liquid fatty phase. Thus, the more the units with the at least one heteroatom increase in polarity and in proportion in the at least one polymer, which corresponds to the presence of several heteroatoms, the greater the affinity of the at least one polymer for polar oils. On the other hand, the more the units with the at least one heteroatom decrease in polarity, indeed even become nonpolar, or in proportion, the greater the affinity of the at least one polymer for nonpolar oils.

The at least one polymer is, for example, a polyamide with a weight-average molecular mass of less than 1,000,000 comprising a) a polymer backbone having amide repeat units and b) optionally at least one optionally functionalized fatty chain chosen from pendent and end fatty chains having from 6 to 120 carbon atoms which are bonded to the amide units.

For example, the at least one pendant fatty chain may be bonded to at least one of the nitrogen atoms of the amide units of the polymer.

For example, the fatty chains of this polyamide ranges from 40% to 98% of the total number of the units with the at least one heteroatom of the at least one polymer such as, from 50% to 95%.

The structuring polymer and, for example, the polyamide of the composition according to the disclosure exhibits a weight-average molecular mass of less than 1,000,000, such as, less than 500,000. For example, the molecular mass may be less than or equal to 100,000 (e.g., ranging from 1000 to 100,000), such as, less than or equal to 50,000 (e.g., ranging from 1000 to 50,000) and further for example, ranging from 1000 to 30,000, such as from 2000 to 20,000 and for example, from 2000 to 10,000.

Mention may be made, to structuring polymers which can be used in the disclosure, of polyamides, for example, branched by pendant fatty chains and/or end fatty chains comprising from 6 to 120 carbon atoms, such as, having from 12 to 120 carbon atoms and further for example, from 12 to 68 carbon atoms, the end fatty chains being bonded to the polymer backbone via bonding groups, such as ester groups.

These polymers may be polymers resulting from a polycondensation between a dicarboxylic acid comprising at least 32 carbon atoms (having, for example, from 32 to 44 carbon atoms) and a diamine having at least 2 carbon atoms (e.g., from 2 to 36 carbon atoms). The diacid is, for example, a dimer of a fatty acid having at least 16 carbon atoms, such as oleic, linoleic or linolenic acid. The diamine is, for example, ethylenediamine, hexylenediamine, or hexamethylenediamine. For polymers comprising one or 2 end carboxylic acid groups, they may be esterified with a monoalcohol having at least 4 carbon atoms, such as from 10 to 36 carbon atoms, further for example, from 12 to 24, such as, from 16 to 24, for example, 18 carbon atoms.

The at least one polymer is, for example, those disclosed in U.S. Pat. No. 5,783,657 of Union Camp. Each of these polymers satisfies, for example, the following formula (I): 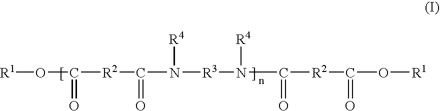
wherein n is a whole number of amide units, such that the number of ester groups ranges from 10% to 50% of the total number of ester and amide groups; R¹ is independently, in each case, chosen from alkyl and alkenyl groups having at least 4 carbon atoms, for example, from 4 to 24 carbon atoms; R² is independently, in each case, chosen from C₄ to C₄₂ hydrocarbonaceous groups, wherein 50% of the R² groups comprise C₃₀ to C₄₂ hydrocarbonaceous groups; R³ is independently, in each case, chosen from organic groups having at least 2 carbon atoms, with hydrogen atoms and optionally with at least one atom chosen from oxygen and nitrogen atoms; and R⁴ is independently, in each case, chosen from hydrogen atoms, C₁ to C₁₀ alkyl groups and direct bonds to R³ or to another R⁴, so that the nitrogen atom to which both R³ and R⁴ are bonded forms part of a heterocyclic structure defined by R⁴—N—R³, with at least 50% of the R⁴ groups comprising a hydrogen atom.

For example, the ester groups of the formula (I), which form part of the end and/or pendant fatty chains within the meaning of the disclosure, comprise from 15% to 40% of the total number of ester and amide groups and, for example, from 20% to 35%. Furthermore, n may comprise an integer ranging from 1 to 10, for example from 1 to 5 and, further for example, greater than 2. For example, R¹ may be a C₁₂ to C₂₂ alkyl group, such as, C₁₆ to C₂₂ alkyl group. R² may be a C₁₀ to C₄₂ hydrocarbonaceous (alkylene) group. For example, 50% at least, such as, 75% of the R² groups are groups having from 30 to 42 carbon atoms. The other R² groups may be C₄ to C₁₉ and even C₄ to C₁₂ hydrogenated groups. For example, R³ comprises a group chosen from C₂ to C₃₆ hydrocarbonaceous groups and polyoxyalkylenated groups and R⁴ comprises a hydrogen atom. For example, R³ comprises a C₂ to C₁₂ hydrocarbonaceous group.

The hydrocarbonaceous groups may be linear, cyclic or branched and saturated or unsaturated groups. Furthermore, the alkyl and alkenyl groups can be linear or branched groups.

According to the disclosure, the structuring (or thickening) of the liquid fatty phase may be obtained using at least one polymer according to the disclosure, for example, of formula (I). The polymers of formula (I) may be generally provided in the form of blends of polymers, it being possible for these blends to further comprise a synthetic product corresponding to a compound of formula (I) wherein n has the value 0, that is to say a diester.

Mention may be made, by way of examples of structuring polymers which may be used in the composition according to the disclosure, of the commercial products manufactured or sold by Arizona Chemical under the names Uniclear 80 and Uniclear 100. They are sold respectively in the form of an 80% (as active material) gel in a mineral oil and a 100% (as active material) gel. They have a softening point of 88° to 94° C. These commercial products are a blend of copolymers of a C₃₆ diacid condensed with ethylenediamine, with an average molecular mass of approximately 6000. The end ester groups result from the esterification of the remaining acid endings with cetyl alcohol, stearyl alcohol or their mixtures (also known as cetearyl alcohol).

Mention may also be made, to structuring polymers which can be used in the disclosure, of polyamide resins resulting from the condensation of an aliphatic dicarboxylic acid and of a diamine (including compounds having more than 2 carbonyl groups and 2 amine groups), the carbonyl and amine groups of adjacent individual units being condensed via an amide bond. These polyamide resins are, for example, those sold under the trade name Versamid® by the companies General Mills Inc. and Henkel Corp. (Versamid 930, 744 or 1655) or by Olin Mathieson Chemical Corp. under the trade name Onamid®, such as Onamid S or C. These resins have a weight-average molecular mass ranging from 6000 to 9000. For further information on these polyamides, reference may be made to U.S. Pat. Nos. 3,645,705 and 3,148,125. Further for example, Versamid® 930 or 744 may be used.

It is also possible to use the polyamides manufactured or sold by Arizona Chemical under the Uni-Rez® references (2658, 2931, 2970, 2621, 2613, 2624, 2665, 1554, 2623, 2662) and the product sold under the reference Macromelt 6212 by Henkel. For further information on these polyamides, reference may be made to U.S. Pat. No. 5,500,209.

It is also possible to use polyamide resins, such as those disclosed in U.S. Pat. Nos. 5,783,657 and 5,998,570.

The structuring polymers of the composition of the disclosure may have a softening temperature of greater than 65° C. and, for example, greater than 70° C. which can range up to 190° C. They, for example, exhibit a softening temperature of less than 150° C., such as ranging from 70° to 140° C. and further for example, ranging from 800 to 130° C. and such as, from 800 to 105° C. These polymers are, for example, nonwaxy polymers. The low melting point of the structuring polymers of the disclsoure facilitates their processing and limits the decomposition of the liquid fatty phase, in contrast to polymers or compounds with a higher softening point.

For example, the polymers of the composition according to the disclosure are those corresponding to formula (I). These polymers may exhibit, because of their fatty chain(s), good solubility in oils and thus, result in macroscopically homogeneous compositions, even with a high level (at least 25%) of polymer, in contrast to polymers without a fatty chain.

As disclosed herein, the softening temperature or melting point values may be determined by the DSC (Differential Scanning Calorimetry) method; the softening temperature or melting point then corresponds to the melting peak and the rise in temperature is 50 or 10° C./min.

The thickening of the at least one fatty phase may be adjusted according to the nature of the at least one polymer and their concentrations and may be such that a viscosity ranging from 1000 to 250,000 cPs and for example, from 10,000 to 50,000 cPs at 25° C., measured with a Rheomat 180 device with a shear rate of 100 s⁻¹, is, for example, obtained.

The at least one polymer as defined above is, for example, present in an amount of ranging from 0.005% to 20% by weight, such as from 0.05% and 10% by weight and, for example, in an amount ranging from 0.05% and 5% by weight and, further for example, from 0.1% and 2% by weight, with respect to the weight of the composition.

As used herein, the term “oil” is understood to mean a liquid fatty substance which is insoluble in water at ambient temperature (25° C.) and atmospheric pressure (760 mm of Hg). The oily phase may be composed of at least one oil which is compatible with one another.

The term “insoluble in water” is understood to mean, within the meaning of the present invention, a substance which exhibits a solubility in pure water of less than 1% at 25° C. and at atmospheric pressure.

The at least one oil used in the present disclosure exhibits a dynamic viscosity at 25° C. of less than 1 Pa·s (1000 cPs), such as ranging from 10⁻³ to 0.1 Pa·s (1 and 100 cPs). The dynamic viscosity may be measured at 25° C. with a shear rate of 100 s⁻¹, for example, with the device referenced Rheomat RM 180 from Mettler.

The at least one oil which can be used in the present dislcosure is, for example, chosen from vegetable oils, mineral oils, synthetic oils, and fatty acid esters.

Mention may, for example, be made, among the vegetable oils which can be used in the present disclosure, of sweet almond oil, avocado oil, castor oil, olive oil, jojoba oil, sunflower oil, wheat germ oil, sesame oil, groundnut oil, grape seed oil, soybean oil, rapeseed oil, safflower oil, coconut oil, maize oil, hazelnut oil, karite butter, palm oil, apricot kernel oil, and calophyllum oil.

Examples of mineral oils are liquid paraffin and liquid petrolatum.

The synthetic oils may be chosen, for example, from polydecenes, squalane, poly(α-olefin)s, such as isododecane and isohexadecane, transesterified vegetable oils, and fluorinated oils.

Use may also be made of fatty acid esters, such as, for example, the compounds of formula RₐCOOR_{b} wherein Rₐ comprises a residue of a higher fatty acid comprising from 5 to 29 carbon atoms and R_{b} comprises a hydrocarbonaceous chain comprising from 3 to 30 carbon atoms, such as purcellin oil (stearyl octanoate), isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate, or 2-octyldodecyl lactate.

The at least one oil, for example, in the context of the present disclosure is chosen from avocado oil, castor oil, olive oil, hydrogenated polydecene, isopropyl myristate, isononyl isononanoate, and liquid paraffin.

The at least one oil as defined above is, for example, present in an amount ranging from 0.01% and 30% by weight, such as in an amount ranging from 0.1% and 15% by weight and, further for example, from 0.1% to 10% by weight, with respect to the weight of the composition, such as, from 0.5% to 5% by weight.

Mention may, for example, be made, as anionic detergent surfactants which can be used, alone or as mixtures, in the context of the present disclosure, of salts, for example, alkali metal salts, such as sodium salts, ammonium salts, amine salts, aminoalcohol salts or alkaline earth metal salts, for example magnesium salts, of the following compounds: alkyl sulphates, alkyl ether sulphates, alkylamido ether sulphates, alkylaryl polyether sulphates, monoglyceride sulphates; alkylsulphonates, alkyl phosphates, alkylamidesulphonates, alkylarylsulphonates, α-olefinsulphonates, paraffinsulphonates; alkyl sulphosuccinates, alkyl ether sulphosuccinates, alkylamide sulphosuccinates; alkyl sulphoacetates; acylsarcosinates; and acylglutamates, wherein the alkyl and acyl groups of all these compounds comprising from 6 to 24 carbon atoms and the aryl group, for example, denoting a phenyl or benzyl group. Use may also be made of C₆-C₂₄ alkyl esters of polyglycosidecarboxylic acids, such as alkyl glucosidecitrates, alkyl polyglycosidetartrates and alkyl polyglycosidesulphosuccinates; alkyl sulphosuccinamates, acyl isethionates and N-acyltaurates, the alkyl or acyl group of all these compounds comprising from 12 to 20 carbon atoms. Mention may also be made, among the anionic surfactants which can still be used, of the acyl lactylates, the acyl group of which comprises from 8 to 20 carbon atoms.

In addition, mention may also be made of alkyl-D-galactosideuronic acids and their salts, and polyoxyalkylenated (C₆-C₂₄)alkyl ether carboxylic acids, polyoxyalkylenated (C₆-C₂₄)alkyl(C₆-C₂₄)aryl ether carboxylic acids, polyoxyalkylenated (C₆₋₂₄)alkylamido ether carboxylic acids and their salts, for example, those comprising from 2 to 50 ethylene oxide groups, and their mixtures.

In one embodiment, the anionic surfactants which may be used in the composition according to the disclosure are salts, such as sodium, magnesium or ammonium salts, of alkyl sulphates; of alkyl ether sulphates, such as sodium lauryl ether sulphate, such as with 2 or 3 mol of ethylene oxide; of alkyl ether carboxylates; and their mixtures, the alkyl groups generally comprising from 6 to 24 carbon atoms and, for example, from 8 to 16 carbon atoms.

The nonionic detergent surface-active agents which may be used in the composition according to the disclosure are compounds well known per se (see, e.g., “Handbook of Surfactants” by M. R. Porter, published by Blackie & Son (Glasgow and London), 1991, pp. 116-178). Thus, the non-ionic detergent surface-active agents may be chosen, for example, from polyethoxylated, polypropoxylated and polyglycerolated fatty acids, (C₁-C₂₀)alkylphenols, α-diols and alcohols having a fatty chain comprising, for example, from 8 to 18 carbon atoms. It being possible for the number of ethylene oxide or propylene oxide groups to range, for example, from 2 to 50 and it being possible for the number of glycerol groups to range, for example, from 2 to 30. Mention may also be made, for example, of copolymers of ethylene oxide and of propylene oxide, condensates of ethylene oxide and of propylene oxide with fatty alcohols; polyethoxylated fatty amides such as having from 2 mol to 30 mol of ethylene oxide, polyglycerolated fatty amides comprising on average from 1 to 5 glycerol groups and, for example, from 1.5 to 4; polyethoxylated fatty amines such as having 2 mol to 30 mol of ethylene oxide; sorbitan ethoxylated fatty acid esters having from 2 mol to 30 mol of ethylene oxide; sucrose fatty acid esters, polyethylene glycol esters of fatty acids, (C₆-C₂₄)alkylpolyglycosides, N-(C₆-C₂₄)alkylglucamine derivatives, amine oxides, such as oxides of (C₁₀-C₁₄)alkylamines or N-(C₁₀-C₁₄)acylaminopropylmorpholine oxides; and their mixtures.

Use is, for example, made, among the abovementioned nonionic surfactants, of (C₆-C₂₄)alkylpolyglycosides such as, (C₈-C₁₆)alkylpolyglycosides.

The at least surfactant is present in an amount ranging from 3% and 50% by weight, such as, from 4% and 30% by weight, for example, from 5% and 20% by weight, with respect to the total weight of the detergent composition.

The detergent composition according to the dislcosure can further comprise at least one amphoteric surfactant and optionally nondetergent surfactants, for example, nonionic nondetergent surfactants.

The at least one amphoteric surface-active agents, i.e., amphoteric surfactants, which are suitable in the present disclosure may be, for example, derivatives of aliphatic secondary or tertiary amines wherein the aliphatic group is a linear or branched chain comprising 8 to 22 carbon atoms and comprising at least one water-solubilizing anionic group, such as, for example, a carboxylate, sulphonate, sulphate, phosphate or phosphonate group; mention may also be made of (C₈-C₂₀)alkylbetaines, sulphobetaines, (C₈-C₂₀)alkylamido(C₆-C₈)alkylbetaines or (C₈-C₂₀)alkylamido(C₆-C₈)alkylsulphobetaines; and their mixtures.

Mention may be made, among the amine derivatives, of the products sold under the name Miranol®, as disclosed in U.S. Pat. Nos. 2,528,378 and 2,781,354 and classified in the CTFA dictionary, 3rd edition, 1982, under the names Amphocarboxyglycinate and Amphocarboxypropionate, with the respective structures (2) and (3):
R₂—CONHCH₂CH₂—N⁺(R₃)(R₄)(CH₂COO⁻) (2)
wherein:
R₂ is at least one group chosen form alkyl groups derived from an acid R₂—COOH present in hydrolysed coconut oil, and heptyl, nonyl and undecyl groups,
R₃ is a β-hydroxyethyl group and
R₄ is a carboxymethyl group; and
   R_{2′}—CONHCH₂CH₂—N(B)(C) (3)
wherein:
B is —CH₂CH₂OX′,
C is —(CH₂)_{z}—Y′, with z=1 or 2,
X′ is an entity chosen from a —CH₂CH₂—COOH group and a hydrogen atom,
Y′ is a group chosen from —COOH and —CH₂—CHOH—SO₃H groups,
R_{2′} is at least one group chosen from alkyl groups of an acid R_{2′}—COOH present in coconut oil and in hydrolysed linseed oil, alkyl groups, such as C₁₇ alkyl groups and its iso form, and unsaturated C₁₇ groups.

These compounds are classified in the CTFA dictionary, 5th Edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphodipropionate, Disodium Caprylamphodipropionate, Disodium Capryloamphodipropionate, Lauroamphodipropionic acid, and Cocoamphodipropionic acid.

Mention may be made, by way of example, of the cocoamphodiacetate sold by Rhodia under the trade name Miranol® C2M concentrate.

Use is, for example, made, among the amphoteric surfactants, of (C₈-C₂₀)alkylbetaines, (C₈-C₂₀)alkylamido(C₆-C₈)alkylbetaines, alkylamphodiacetates, and their mixtures.

The at least one amphoteric surfactant is generally present in an amount ranging from 0.1% and 20% by weight, such as from 0.5% and 15% by weight, further for example, from 1% to 10% by weight, with respect to the total weight of the detergent composition.

The composition according to the disclosure, may, for example, further comprise at least one cationic polymer. As used herein, the term “cationic polymer” is understood to mean any polymer comprising cationic groups and/or groups which may be ionized to give cationic groups.

The at least one cationic polymer which may be used in accordance with the present disclosure may be chosen from all those already known per se as improving the cosmetic properties of hair treated with detergent compositions, namely, for example, those disclosed in European Patent Application No. 0 337 354 and in French Patent Application Nos. 2 270 846, 2 383 660, 2 598 611, 2 470 596, and 2 519 863.

In one embodiment, the at least one cationic polymer is chosen from those which comprise units comprising primary, secondary, tertiary, and quaternary amine groups which can either form part of the main polymer chain or be carried by a side substituent directly connected to the main chain.

The at least one cationic polymer used generally has a number-average molecular mass ranging from 500 and 5×10⁶ approximately and, further for example, from 10³ and 3×10⁶ approximately.

Mention be made, further for example, among cationic polymers, of the polymers of the polyamine, polyaminoamide, and poly(quaternary ammonium) type. These are known products.

The polymers of the polyamine, polyaminoamide, or poly(quaternary ammonium) type which may be used in the composition of the present disclosure are those disclosed in French Patents Nos. 2,505,348 and 2,542,997. Mention may be made, among these polymers, of:

(1) Homopolymers or copolymers derived from acrylic or methacrylic acid esters or amides.

(2) Cellulose ether derivatives comprising quaternary ammonium groups disclosed in French Patent No. 1,492,597.

(3) Cationic cellulose derivatives, such as cellulose copolymers or cellulose derivatives grafted with a water-soluble quaternary ammonium monomer and disclosed, for example, in U.S. Pat. No. 4,131,576, such as hydroxyalkylcelluloses, for example hydroxymethyl-, hydroxyethyl- or hydroxypropylcelluloses, grafted such as with a methacryloyloxyethyltrimethylammonium, methacrylamidopropyltrimethylammonium, or dimethyldiallylammonium salt.

(4) Cationic polysaccharides disclosed, for example, in U.S. Pat. Nos. 3,589,578 and 4,031,307, such as guar gums comprising trialkylammonium cationic groups.

(5) Polymers comprised of piperazinyl units and of divalent, straight- or branched-chain alkylene or hydroxyalkylene groups, optionally interrupted by oxygen, sulfur or nitrogen atoms or by aromatic or heterocyclic rings, as well as the oxidation and/or quaternization products of these polymers. Such polymers are disclosed, for example, in French Patent Nos. 2,162,025 and 2,280,361.

(6) Water-soluble polyaminoamides, such as those disclosed, for example, in French Patent Nos. 2,252,840 and 2,368,508.

(7) Polyaminoamide derivatives, for example, adipic acid/dialkylaminohydroxyalkyldialkylenetriamine polymers wherein the alkyl group comprises from 1 to 4 carbon atoms and, for example, denotes a methyl, ethyl or propyl group and the alkylene group comprises from 1 to 4 carbon atoms and, for example, comprises the ethylene group. Such polymers are disclosed, for example, in French Patent No. 1,583,363.

(8) Polymers obtained by reaction of a polyalkylenepolyamine comprising two primary amine groups and at least one secondary amine group with a dicarboxylic acid chosen from diglycolic acid and saturated aliphatic dicarboxylic acids having from 3 to 8 carbon atoms, the molar ratio of polyalkylenepolyamine to dicarboxylic acid ranges from 0.8:1 to 1.4:1; the polyaminoamide resulting therefrom being reacted with epichlorohydrin in a molar ratio of epichlorohydrin in relation to the secondary amine group of the polyaminoamide ranging from 0.5:1 to 1.8:1. Such polymers are disclosed, for example, in U.S. Pat. Nos. 3,227,615 and 2,961,347.

(9) Cyclopolymers of alkyldiallylamine or of dialkyldiallylammonium, such as homopolymer of dimethyldiallylammonium chloride and copolymers of diallyidimethylammonium chloride and of acrylamide.

(10) Quaternary diammonium polymers exhibiting a number-average molecular mass generally range from 1000 to 100,000, such as those disclosed, for example, in French Patent Nos. 2,320,330, 2,270,846, 2,316,271, 2,336,434, and 2,413,907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945, and 4,027,020.

(11) Poly(quaternary ammonium) polymers, such as those disclosed, for example, in European Patent Application No. 122 324.

(12) Quaternary polymers of vinylpyrrolidone and of vinylimidazole, such as, for example, the products sold by BASF under the names Luviquat® FC 905, FC 550 and FC 370.

(13) Polyamines, such as Polyquart® H sold by Henkel, referenced under the name of “Polyethylene Glycol (15) Tallow Polyamine” in the CTFA dictionary.

(14) Crosslinked polymers of methacryloyloxy(C₁-C₄)alkyltri(C₁-C₄)alkylammonium salts, such as those sold by Allied Colloids under the names of Salcare® SC 92, Salcare® SC 95, and Salcare® SC 96; and their mixtures.

Other cationic polymers which can be used in the context of the disclosure are cationic proteins or cationic protein hydrolysates, polyalkyleneimines, for example, polyethyleneimines, polymers comprising vinylpyridine or vinylpyridinium units, condensates of polyamines and of epichlorohydrin, quaternary polyureylenes and chitin derivatives.

Use may, for example, be made, among the cationic polymers mentioned above which are suitable in the disclosure, of quaternary cellulose ether derivatives, cationic guar gums, cationic cyclopolymers, quaternary polymers of vinylpyrrolidone and of vinylimidazole, and their mixtures.

The at least one cationic polymer is generally present in an amount ranging from 0.01% to 20%, such as from 0.05% to 10% and, for example, from 0.1 to 5% by weight, with respect to the total weight of the composition.

As used herein, the term “cosmetically acceptable medium” is understood to mean a medium compatible with keratinous substances, for example, the hair, but also with a pleasant smell, a pleasing appearance, and a pleasant touch.

The cosmetically acceptable medium comprises water or a mixture of water and of a cosmetically acceptable solvent chosen from lower C₁-C₄ alcohols, such as ethanol, isopropanol, tert-butanol or n-butanol; polyols, such as glycerol, propylene glycol and polyethylene glycol; and their mixtures.

The composition, for example, comprises from 70% to 95% by weight of water with respect to the total weight of the composition.

The washing compositions according to the disclosure exhibit a final pH generally ranging from 3 and 10. For example, this pH ranges from 4.5 to 8. The pH may be conventionally adjusted to the desired value by addition of a base (organic or inorganic) to the composition, for example aqueous ammonia or a primary, secondary or tertiary (poly)amine, such as monoethanolamine, diethanolamine, triethanolamine, isopropanol-amine or 1,3-propanediamine, or alternatively by addition of an acid, preferably a carboxylic acid, such as, for example, citric acid.

The compositions in accordance with the disclosure can comprise, in addition to the combination defined above, viscosity-regulating agents, such as electrolytes, or thickening agents (associative or nonassociative). Mention may, for example, be made of sodium chloride, sodium xylenesulphonate, scleroglucans, xanthan gums, fatty acid alkanolamides, alkyl ether carboxylic acid alkanolamides optionally oxyethylenated with up to 5 mol of ethylene oxide, such as the product sold by Chem Y under the name “Aminol A15”, crosslinked poly(acrylic acid)s and crosslinked copolymers of acrylic acid, such as copolymers of acrylic acid and of C₁₀-C₃₀ alkyl acrylates. These viscosity-regulating agents are used in the compositions according to the disclsoure in proportions which can range up to 10% by weight, with respect to the total weight of the composition.

The compositions in accordance with the disclosure may also comprise, for example, up to 5% of pearlescence or opacifying agents well known in the state of the art, such as, for example, greater than C₁₆ fatty alcohols, acylated derivatives with a fatty chain, such as ethylene glycol or polyethylene glycol monostearates or distearates, or ethers with fatty (C₁₀-C₃₀) chains, such as, for example, distearyl ether or 1-(hexadecyloxy)-2-octadecanol.

The compositions in accordance with the disclosure can optionally also comprise at least one additive chosen from foam synergists, such as C₁₀-C₁₈ 1,2-alkanediols and fatty alkanolamides derived from mono- and from diethanolamine, silicone and non-silicone sunscreens, cationic surfactants, proteins, protein hydrolysates, ceramides, pseudoceramides, fatty acids with linear and branched C₁₂-C₄₀ chains, such as 18-methylicosanoic acid, hydroxy acids, vitamins, such as for example, vitamins E, C and B, provitamins, such as panthenol, silicones, animal, mineral or synthetic oils, anionic, cationic, nonionic, amphoteric or zwitterionic polymers different from polymers with at least one heteroatome according to the disclosure, UV screening agents, fragrances, colorants, natural and synthetic thickeners, C₁₂-C₃₀ fatty alcohols, pearlescence agents, preservatives, pH stabilizing agents, antimicrobial agents, agents for combatting dandruff, antiseborrhoeic agents, antioxidants and reducing agents, and acidic and alkaline agents and any other additive conventionally used in the cosmetics field which does not affect the properties of the composition according to the invention and their mixtures.

The washing compositions according to the disclosure may, of course, further comprise any conventional adjuvant encountered in the field of shampoos, such as, for example, fragrances, preservatives, sequestering agents, softeners, colorants, moisturizing agents, agents for combating dandruff, antiseborrhoeic agents, agents for combating hair loss, and others.

Of course, a person skilled in the art will take care to choose this or these optional additional compounds and/or their amounts so that the advantageous properties intrinsically attached to the combination in accordance with the disclosure are not, or not substantially, detrimentally affected by the envisaged addition or additions.

These compositions may be provided in a form chosen from liquids, optionally thickened liquids, creams and gels and these compositions may be suitable mainly for washing the hair.

These compositions may be provided in the form of an oil-in-water emulsion.

The compositions may be packaged in various forms, for example in bottles, pump-action sprays or aerosol containers, in order to provide for application of the composition in the vaporized form or in the foam form. Such packaging forms are indicated, for example, when it is desired to obtain a foam for treating the hair.

The present disclosure also relates to a process for the cosmetic treatment of keratinous substances comprising applying, to the keratinous substances, an effective amount of a composition as described above and in rinsing, after an optional leave-in time.

The compositions in accordance with the disclosure may be used as shampoos or shower gels, for example, as shampoos for the hair.

When the compositions in accordance with the disclosure are employed as conventional shampoos, they are simply applied to wet hair and the foam generated by massaging or rubbing with the hands is subsequently removed, after an optional leave-in time, by rinsing with water, it being possible for the operation to be repeated at least one time.

Other than in the operation examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that numerical ranges and parameters setting forth the broad scope of the disclosure are approximations, the numerical values set forth in the specific examples are reported to as precisely as possible. Any numerical value, however, inherently contains certain errors necessarily resulting from the standard deviation found in their respective testing measurements.

The following examples illustrate the present disclosure.

### EXAMPLE 1

Mixtures of polymers with the oils shown in the table below were prepared in the ratios shown below:

| | | Oil/polymer | |
|---|---|---|---|
| Oil | Polymer | ratio | Viscosity |
| Castor oil | Copolymer of a C₃₆ diacid | 90/10 | 9500 cPs |
| Isononyl | condensed with | 90/10 | 14 500 cPs |
| isononanoate | ethylenediamine (Uniclear | | |
| | 100VG from Arizona | | |
| | Chemical) | | |

### EXAMPLE 2

The following shampoo was prepared with the thickened oil described in Example 1. The proportions were shown as % by weight.

| Composition | Ex. 2 | |
|---|---|---|
| 70% Sodium lauryl ether sulphate | 14 | g AM |
| 30% Cocobetaine | 2.8 | g AM |
| Cocamide MIPA⁽¹⁾ | 1.5 | g AM |
| Carbomer⁽²⁾ | 0.2 | g |
| Isononyl isononanoate | 2.83 | g |
| Uniclear 100VG | 0.17 | g AM |
| Hydroxystearyl cetyl ether and cetyl alcohol | 2.5 | g |
| pH agent q.s. | pH 7 | |
| Preservative q.s. | | |
| Water q.s. for | 100 | g |

AM: Active Material
⁽¹⁾sold by Huntsman under the trade name Empilan CIS
⁽²⁾sold by Noveon under the trade name Carbopol 980

This composition, applied to natural hair, conferred, after a leave-in time of 3 minutes and rinsing, an excellent conditioning effect on the hair.

1. A composition comprising, in a cosmetically acceptable aqueous medium,
at least one oil,
at least one surfactant chosen from anionic and nonionic detergent surfactants,
at least one polymer with a weight-average molecular mass of less than or equal to 1,000,000 comprising a) a polymer backbone comprising hydrocarbonaceous repeat units provided with at least one heteroatom and b) at least one optionally functionalized fatty chain chosen from pendent and end fatty chains having from 6 to 120 carbon atoms bonded to the hydrocarbonaceous units, and
from 70% to 95% by weight of water with respect to the total weight of the composition.

2. The composition according to claim 1, further comprising at least one amphoteric surfactant.

3. The composition according to claim 1, wherein the average molecular mass of the at least one polymer is less than or equal to 500,000.

4. The composition according to claim 3, wherein the average molecular mass of the at least one polymer is less than or equal to 100,000.

5. The composition according to claim 1, wherein the hydrocarbonaceous repeat units provided with the at least one heteroatom comprise at least one nitrogen atom.

6. The composition according to claim 1, wherein the hydrocarbonaceous repeat units provided with the at least one heteroatom are amide groups.

7. The composition according to claim 1, wherein from 40% to 98% of the number of units of fatty chains are from units with the hydrocarbonaceous repeat units provided with at least one heteroatom and the at least one optionally functionalized fatty chain.

8. The composition according to claim 7, wherein from 50% to 95% of the number of units of fatty chains are from units with the hydrocarbonaceous repeat units provided with at least one heteroatom and the at least one optionally functionalized fatty chain.

9. The composition according to claim 1, wherein the at least one optionally functionalized fatty chain comprises pendant fatty chains bonded directly to the at least one heteroatom.

10. The composition according to claim 3, wherein the at least one optionally functionalized fatty chain comprises pendant fatty chains bonded directly to at least one of the nitrogen atoms of the amide units.

11. The composition according to claim 1, wherein the weight-average molecular mass ranges from 1000 to 30,000.

12. The composition according to claim 11, wherein the weight-average molecular mass ranges from 2000 to 10,000.

13. The composition according to claim 1, wherein the at least one optionally functionalized fatty chain comprises end fatty chains bonded to the polymer backbone via bonding groups.

14. The composition according to claim 13, wherein the bonding groups are ester groups.

15. The composition according claim 1, wherein the at least one optionally functionalized fatty chain comprises from 12 to 68 carbon atoms.

16. The composition according to claim 1, wherein the at least one polymer is chosen from polymers of formula (I) and mixtures thereof: 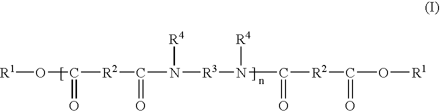
wherein n is a number of amide units, such that the number of ester groups comprise from 10% to 50% of the total number of ester and amide groups; R¹ is independently, in each case, chosen from alkyl and alkenyl groups having at least 4 carbon atoms; R² is independently, in each case, chosen from C₄ to C₄₂ hydrocarbonaceous groups, wherein 50% of the R² groups comprises C₃₀ to C₄₂ hydrocarbonaceous groups; R³ is independently, in each case, chosen from organic groups having at least 2 carbon atoms, with hydrogen atoms and optionally at least one atom chosen from oxygen and nitrogen atoms; and R⁴ is independently, in each case, chosen from hydrogen atoms, C₁ to C₁₀ alkyl groups and direct bonds to R³ or to another R⁴, wherein the nitrogen atom to which both R³ and R⁴ are bonded forms part of a heterocyclic structure defined by R⁴—N—R³, with at least 50% of the R⁴ groups comprising a hydrogen atom.

17. The composition according to claim 1, wherein R¹ is a C₁₂ to C₂₂ alkyl group.

18. The composition according to claim 16, wherein R² are groups having from 30 to 42 carbon atoms.

19. The composition according to claim 1, wherein the at least one polymer is present in an amount ranging from 0.005% to 20% by weight, with respect to the total weight of the composition.

20. The composition according to claim 19, wherein the at least one polymer is present in an amount ranging from 0.05% to 10% by weight, with respect to the total weight of the composition.

21. The composition according to claim 1, wherein the at least one oil is chosen from vegetable oils, mineral oils, synthetic oils, and fatty acid esters.

22. The composition according to claim 21, wherein the at least one oil is chosen from sweet almond oil, avocado oil, castor oil, olive oil, jojoba oil, sunflower oil, wheat germ oil, sesame oil, groundnut oil, grape seed oil, soybean oil, rapeseed oil, safflower oil, coconut oil, maize oil, hazelnut oil, karite butter, palm oil, apricot kernel oil and calophyllum oil; liquid paraffin and liquid petrolatum; polydecenes, squalane, poly(α-olefin)s, transesterified vegetable oils and fluorinated oils; and compounds of formula RₐCOOR_{b} wherein Rₐ comprises a residue of a higher fatty acid comprising from 5 to 29 carbon atoms and R_{b} comprises a hydrocarbonaceous chain comprising from 3 to 30 carbon atoms.

23. The composition according to claim 22, wherein the at least one oil is chosen from avocado oil, castor oil, olive oil, isohexadecane, polydecene, isopropyl myristate, isononyl isononanoate, and liquid paraffin.

24. The composition according to claim 1, wherein the at least one oil is present in an amount ranging from 0.01% to 30% by weight, with respect to the total weight of the composition.

25. The composition according to claim 24, wherein the at least one oil is present in an amount ranging from 0.1% to 15% by weight, with respect to the total weight of the composition.

26. The composition according to claim 1, wherein the at least one oil is prethickened by the polymer backbone having hydrocarbonaceous repeat units provided with at least one heteroatom.

27. The composition according to claim 1, wherein the at least one oil/the at least one polymer comprises a heteroatom ratio by weight greater than or equal to 50/50.

28. The composition according to claim 27, wherein the at least one oil/the at least one polymer comprises a heteroatom ratio by weight greater than or equal to 60/40.

29. The composition according to claim 27, wherein the at least one oil/the at least one polymer comprises a heteroatom ratio by weight ranging from 60/40 to 99/1.

30. The composition according to claim 1, wherein the at least one oil exhibits a number-average primary size ranging from 1 μm to 100 μm.

31. The composition according to claim 30, wherein the at least one oil exhibits a number-average primary size ranging from 5 μm to 30 μm.

32. The composition according to claim 1, wherein the anionic surfactants are chosen from alkali metal salts, ammonium salts, amine salts, aminoalcohol salts and alkaline earth metal salts of at least one compound chosen from compounds: alkyl sulphates, alkyl ether sulphates, alkylamido ether sulphates, alkylaryl polyether sulphates, monoglyceride sulphates; alkylsulphonates, alkyl phosphates, alkylamidesulphonates, alkylarylsulphonates, α-olefinsulphonates, paraffinsulphonates; alkyl sulphosuccinates, alkyl ether sulphosuccinates, alkylamide sulphosuccinates; alkyl sulphoacetates; acylsarcosinates; and acylglutamates, wherein the alkyl and acyl groups of the salts comprise from 6 to 24 carbon atoms and the aryl groups comprise groups chosen from phenyl and benzyl groups.

33. The composition according to claim 32, wherein the anionic surfactants are chosen from C₆-C₂₄ alkyl esters of polyglycosidecarboxylic acids; and alkyl sulphosuccinamates, acyl isethionates and N-acyltaurates, wherein the alkyl and acyl groups comprise from 12 to 20 carbon atoms.

34. The composition according to claim 32, wherein the anionic surfactants are chosen from salts of alkyl sulphates, salts of alkyl ether sulphates, optionally with ethylene oxide ranging from 2 mol to 3 mol, and salts of alkyl ether carboxylates, wherein the alkyl groups comprise from 6 to 24 carbon atoms.

35. The composition according to claim 1, wherein the nonionic surfactants are chosen from polyethoxylated, polypropoxylated and polyglycerolated fatty acids, (C₁-C₂₀)alkylphenols, α-diols and alcohols having a fatty chain; copolymers of ethylene oxide and of propylene oxide, condensates of ethylene oxide and of propylene oxide with fatty alcohols; polyethoxylated fatty amides, polyglycerolated fatty amides comprising on average from 1 to 5 glycerol groups; polyethoxylated fatty amines; sorbitan ethoxylated fatty acid esters having from 2 mol to 30 mol of ethylene oxide; and sucrose fatty acid esters, polyethylene glycol esters of fatty acids, (C₆-C₂₄)alkylpolyglycosides, N-(C₆-C₂₄)alkylglucamine derivatives, amine oxides and N-(C₁₀-C₁₄)acylaminopropylmorpholine oxides.

36. The composition according to claim 35, wherein the nonionic surfactants are chosen from (C₆-C₂₄)alkylpolyglycosides.

37. The composition according to claim 36, wherein the nonionic surfactants are chosen from (C₈-C₁₆)alkylpolyglycosides.

38. The composition according to claim 1, wherein the at least one surfactant is present in an amount ranging from 3% to 50% by weight, with respect to the total weight of the composition.

39. The composition according to claim 38, wherein the at least one surfactant is present in an amount ranging from 4% to 30% by weight, with respect to the total weight of the composition.

40. The composition according to claim 2, wherein the at least one amphoteric surfactant is chosen from (C₈-C₂₀)alkylbetaines, (C₈-C₂₀)alkylamido(C₆-C₈)alkylbetaines, and alkylamphodiacetates.

41. The composition according to claim 2, wherein the at least one amphoteric surfactant is present in an amount ranging from 0.1% to 20% by weight, with respect to the total weight of the composition.

42. The composition according to claim 41, wherein the at least one amphoteric surfactant is present in an amount ranging from 0.5% to 15% by weight, with respect to the total weight of the composition.

43. The composition according to claim 1, wherein the cosmetically acceptable medium comprises water and a cosmetically acceptable solvent.

44. The composition according to claim 43, wherein the cosmetically acceptable solvent is chosen from lower C₁-C₄ alcohols, polyols, and their mixtures.

45. The composition according to claim 1, further comprising at least one conventional additive chosen from cationic surface-active agents; anionic, cationic, nonionic, amphoteric and zwitterionic polymers; UV screening agents; fragrances; colorants; natural and synthetic thickeners; C₁₂-C₃₀ fatty alcohols; pearlescence agents; preservatives; pH stabilizing agents; vitamins; provitamins; antimicrobial agents; agents for combating dandruff; antiseborrhoeic agents; antioxidants and reducing agents; and acidic and alkaline agents.

46. The composition according to claim 1, further comprising at least one cationic polymer.

47. The composition according to claim 46, wherein the at least one cationic polymer is chosen from quaternary cellulose ether derivatives, cationic guar gums, cationic cyclopolymers, quaternary polymers of vinylpyrrolidone and vinylimidazole, and their mixtures.

48. The composition according to claim 46, wherein the at least one cationic polymer is present in an amount ranging from 0.01% to 20% by weight, with respect to the total weight of the composition.

49. The composition according to claim 48, wherein the at least one cationic polymer is present in an amount ranging from 0.05% to 10% by weight, with respect to the total weight of the composition.

50. The composition according to claim 49, wherein the at least one cationic polymer is present in an amount ranging from 0.1% to 5% by weight, with respect to the total weight of the composition.

51. The composition according to claim 1, wherein the composition is in the form of an oil-in-water emulsion.

52. A process for treating hair comprising
applying, to the hair, an effective amount of a composition comprising, in a cosmetically acceptable aqueous medium, at least one oil, at least one surfactant chosen from anionic and nonionic detergent surfactants, at least one polymer with a weight-average molecular mass of less than or equal to 1,000,000 comprising a) a polymer backbone comprising hydrocarbonaceous repeat units provided with at least one heteroatom and b) at least one optionally functionalized fatty chain chosen from pendent and end fatty chains having from 6 to 120 carbon atoms bonded to the hydrocarbonaceous units, and from 70% to 95% by weight of water with respect to the total weight of the composition and rinsing, after an optional leave-in time.
